(19) United States
(12) Patent Application Publication
(10) Pub. No.: US 2019/0114388 A1
(43) Pub. Date: Apr. 18, 2019

(54) DETECTION, MEASUREMENT, AND ANALYSIS OF DNA REPLICATION SIGNALS

(71) Applicant: GENOMIC VISION SA, Bagneux (FR)

(72) Inventors: Jeremie NICOLLE, Bagneux (FR); Vincent GLAUDIN, Sceaux (FR); Ivan KYRGYZOV, L'Hay-Les-Roses (FR); Aaron BENSIMON, Antony (FR)

(73) Assignee: GENOMIC VISION SA, Bagneux (FR)

(21) Appl. No.: 16/160,003

(22) Filed: Oct. 15, 2018

Related U.S. Application Data
(60) Provisional application No. 62/573,019, filed on Oct. 16, 2017.

(51) Int. Cl. G06F 19/16; C12Q 1/6813; G06F 19/22; G06K 9/00

(52) U.S. Cl. CPC G06F 19/16; C12Q 1/6813; G06K 2209/07; G06K 9/00147; G06K 9/00134; G06F 19/22

(57) ABSTRACT

An automated, computer-implemented method for detecting and measuring large amounts of data obtained from molecular combing procedures including the identification and characterization of seven different DNA replication initiation and termination patterns.

### BACKGROUND

### Field of the Invention

The present invention relates to a method for analyzing large quantities of image data that describe DNA replication in stretched or combed labelled purified genomic fragments or in other cellular, nuclear or mitochondrial DNA fragments. These data are obtained by molecular combing or other methods that provide linear DNA. Large quantities of such image data may be present in a data square having a pixel length of 50,000, 100,000 or 150,000 pixels or more.

Image data is obtained from replicating DNA that has been differentially labeled using nucleotides tagged with different colored dyes, for example, replicating DNA may be pulse labeled with nucleotide coupled to a green dye labeling segments of replicating DNA fibers green and then subsequently pulse labeled with a nucleotide coupled to a red dye that identifies segments of DNA where replication has been initiated after addition of the second red dye label. To help identify DNA strands that are not labeled either green or red (e.g., where replication has not initiated or occurred) the unlabeled strands may be counterstained, for example, with a blue dye. The replicating DNA is then subjected to molecular combing and subsequent recognition of labelling patterns, e.g., in the example above, patterns of green, red and blue.

The inventors identify at least seven different replication patterns which describe initiation and termination points of DNA replication using this procedure. The invention identifies differentially colored (e.g., green, red and blue) segments in molecules of replicating DNA, removes image noise and artifacts, and recognizes, characterizes and quantifies particular DNA replication initiation and termination patterns or signals. It can quickly process and data mine large quantities of image data that are not feasible or impossible to analyze manually. A preferred aspect of the invention concerns the use of a computer-implemented method for the analysis of data obtained from selected DNA fragments or their replicating segments including steps of data acquisition, detection of signals, pattern characterization, selection of particular signals, and measurement, quantification, and/or other analysis of replication signal data.

The present invention also relates to a method for analyzing the images of DNA replication events to evaluate DNA replication activity in various circumstances. Such circumstances include evaluating the effect of DNA replication-inhibiting drugs such as cisplatin/carboplatin which perturb progress of replication forks and cdc7 inhibitors which interfere with initiation of DNA replication.

The invention can be used to diagnose the presence or absence of a genetic defect affecting a DNA replication pathway. This is advantageous for assessing gene therapy that corrects a defect as DNA replication in the modified cells can be characterized and compared to that in normal cells.

A preferred aspect of the invention concerns the use of a computer-implemented method for the analysis of selected DNA fragments or regions involved during the replication mechanism, comprising steps of detection, selection, measurement, and analysis of replicated signals and target regions.

### Description of the Related Art

The “background” description provided herein is for the purpose of generally presenting the context of the disclosure. Work of the presently named inventor(s), to the extent it is described in this background section, as well as aspects of the description which may not otherwise qualify as prior art at the time of filing, are neither expressly or impliedly admitted as prior art against the present invention.

DNA Replication.

Control of DNA synthesis is essential for maintaining genome stability (Litmanovitch, T.; Altaras, M. M.; Dotan, A.; Avivi, L.; Cytogenetics & Cell Genetics. 81(1):26-35, 1998; Schimke, R. T.; Sherwood, S. W.; Hill, A. B.; Johnston, R. N.; Proceedings of the National Academy of Sciences of the United States of America. 83(7):2157-61, 1986; Auerbach, A. D.; Verlander P C. Current Opinion in Pediatrics. 9(6):600-16, 1997). In higher eukaryotes, genomic instability associated with a loss of replication control and aberrant DNA synthesis is a key feature of a variety of neoplasms and genetic diseases (Amiel, A.; Litmanovitch, T.; Lishner, M.; Mor, A.; Gaber, E.; Tangi, I.; Fejgin, M.; Avivi, L.; Genes, Chromosomes & Cancer. 22(3):225-31, 1998; Amiel, A.; Litmanovich, T.; Gaber, E.; Lishner, M.; Avivi, L.; Fejgin, M. D.; Human Genetics. 101(2):219-22, 1997; Weksberg, R.; Squire, J. A.; Molecular biology of Beckwith-Wiedemann syndrome. (Review] [65 refs] [Journal Article. Review. Review, Tutorial Medical & Pediatric Oncology. 27 (5): 4 62-9, 1996). In yeast, an altered pattern of DNA synthesis leads to genomic abnormalities including aneuploidies and translocations (Elledge, S. J.; Science. 274(5293):1664-72, 1996 Stillman, B.; Cell cycle control of DNA replication. Science. 274(5293):1659-64, 1996; Morrow, D. M.; Connelly, C.; Hieter, P.; Genetics. 147(2):371-82, 1996).

Origins of Replication.

Eukaryotic genomes are duplicated by the activation of multiple bidirectional origins of replication. The replication programs of these cells depend on the temporal and spatial organization of replication origins throughout the genome.

The temporal and spatial pattern of activation or replication program varies according to the developmental stage (Hand, R.; Eucaryotic DNA: organization of the genome for replication. Cell. 15(2):317-25, 1978. Hyrien, O.; Maric, C.; Mechali, M.; Transition in specification of embryonic meazroan DNA replication origins. Science. 270(5238):994-7, 1995). In Xenopus laevi, for example, the duration of the period of DNA replication during the cell cycle depends upon replicon size or on the distribution of replication origins (Walter, J.; Newport, J. W.; Science. 2-75(5302):993-5, 1997). However, the organization and distribution of replication origins throughout the eukaryotic genome is not well known and consequently the regulation of the replication program in these cells is poorly understood (Diffley, J. F.; Once and only once upon a time: specifying and regulating origins of DNA replication in eukaryotic cells. Genes & Development. 10(22):2819-30, 1996). This is primarily due to a variety of technical and fundamental obstacles which make it difficult to study DNA replication at the genomic level. While a number of techniques exist for studying DNA replication in both higher and lower eukaryotes, more rapid methods are needed for the quantitative analysis of the dynamics of genome duplication (DePamphilis, N. L.; Methods. 13(3):211-9, 1997).

Molecular Combing.

Molecular combing techniques are known in the art, including those incorporated by reference to Bensimon, et al., U.S. Pat. No. 6,248,537 B1 and to Bensimon, et al., EP 1 192 283 B1. As shown herein, molecular combing now has been applied to study DNA replication. Replicating DNA is differentially labeled at successive time points after the beginning of DNA synthesis, the DNA is extracted and combed on a glass surface, and labelling patterns visualized.

Some molecular combing procedures involve the use of mapping probes, such as those described and incorporated by reference to Bensimon, et al., U.S. Pat. No. 6,248,537. These probes, for example, can be used to identify particular genetic loci in combed genomic DNA. However, as shown herein such mapping probes and procedures are unnecessary for many parameters of DNA replication such as replication fork speed, inter-origin distance as whole genome information which can be determined without use of such probes. However, when one would like to focus analysis of DNA replication on certain loci of the genome or localization origins of DNA replication in or around such loci, such mapping probes and procedures may be combined with the procedures described herein for DNA replication labelling.

In the methods described herein, detected signals appear as linear fluorescent signals, which result from intermediates produced by incorporation of nucleotides tagged with different colored dyes during DNA replication. In situations where analysis of DNA replication is focused on certain loci, additional signals from labeled probes hybridized to the replicating or replicated DNA in or around the loci of interest may also be detected and analyzed.

Processing of Image Data.

Images of signals can be acquired, analyzed and mapped manually or at high resolution using software developed in the past. However, existing algorithms are too slow and costly. Long wait times of twenty minutes or more and high computational costs are incurred when molecular combing images are very large, for example, for scanner images with pixel length squares of more than 50,000, 100,000 or 200,000. Usually, image data from molecular combing contains tens of thousands or more signals, which makes a manual classification and measurement of all signals impossible. Practically, when manual analysis is performed, only around 500 to 1,000 signals are manually labelled that can take a half of a day and the complete labelling of several thousands of signals can take several days of work for one single biological condition on one image. Comprehensive manual analysis of data from DNA replication images derived from molecular combing procedures, when feasible, is time-consuming and expensive. The full and careful viewing of a 100,000 pixel length scanned image takes about 4 to 6 hours. Furthermore, DNA replication analysis must be performed on several conditions and several images in order to compare obtained results to reference conditions, thus several weeks of work are needed if the analysis is performed manually.

Pattern recognition and contour-shaping algorithms are known, for example, in methods for fingerprint analysis or human face recognition, for example, Benini, U.S. Pat. No. 9,646,197 discloses biometric identification and verification methods and systems using pattern recognition. However, these methods are unsuitable for processing image data from molecular combing procedures due to significant differences in the kind of image data, patterns to be recognized, and particular artifacts associated with DNA labeling and molecular combing.

Methods exist to process biological information containing data and images, such as diagnostic test information or information from high-throughput biological screening procedures, which is often machine-processed. For example, Schoenmeyer, et al., U.S. Pat. No. 9,714,112 B2, describes analysis of diagnostic images; Kapur, et al., U.S. Pat. No. 7,476,510, describes analysis of cellular data obtained from high throughput screening of cells; Proffitt et. al., Cytometry 24:204-213, (1996) describe a semi-automated fluorescence digital imaging system for quantifying relative cell numbers in situ; and the ARRAYSCAN™ System, Dunley, et al., U.S. Pat. No. 5,989,835 and Giuliano, et al., U.S. Pat. No. 6,416,959 describe optical systems for determining the distribution, environment, or activity of luminescently labeled reporter molecules on or in cells for the purpose of screening large numbers of compounds for specific biological activity. These procedures are also unsuitable for processing image data from molecular combing procedures due to significant differences in the kind of image data and differences in associated artifacts.

Manual analysis of DNA replication information from molecular combing has been described in Técher, et al., Replication dynamics: biases and robustness of DNA fiber analysis. Journal of Molecular Biology, 2013, vol. 425, no 23, p. 4845-4855 (incorporated by reference).

The template matching algorithms described by Lewis, et al., Vision Interface, pp. 120-123 (1995) (incorporated by reference) are unsuitable for measuring the lengths of DNA replication patterns obtained from molecular combing because they include steps that decrease the precision of object contour shape detection. Mathematical morphology operations algorithms such as those described by Zana, et al., IEEE Transactions on Image Processing 10(7):1010-1019 (2001) (incorporated by reference) lack the robustness necessary to detect patterns having particular shapes. Moreover, the methods described by Lewis, et al. or Zana, et al. were designed for analysis of natural images coming from a conventional camera.

Methods for segmenting areas in images that are homogeneous in terms of colors and texture characteristics are described by Shi, IEEE Transactions on Pattern Analysis and Machine Intelligence 22(8)888 (2000) and by Vese, et al., Int. J. Computer Vision 50(3):271 (2002) (both incorporated by reference). However, these methods are computationally complex and cannot be applied in adequate timing on large combing images that are squares of more than 100,000 (or 100 k) pixel side size. Moreover, these methods do not concretely address significant technical problems associated with analysis of image data obtained from molecular combing.

When dealing with molecular combing images and practical time constraints it was impossible to use previous computationally complex methods and pattern recognition methods based on machine learning. Previous methods can be separated in three groups aiming at roughly locating a bounding box around the object, classifying objects, and precise localization of landmarks defining the contour and the main elements of the considered object. Most currently used methods are based on deep convolutional neural networks; see Krizhevsky, Alex, Sutskever, Ilya, and Hinton, Geoffrey, E., Imagenet classification with deep convolutional neural networks. In: Advances in neural information processing systems. 2012. p. 1097-1105.

These previous computational complex approaches were not practically feasible due to the large numbers of objects that must be detected in a molecular combing image, as well as the large numbers of areas that are to be given as inputs to these object-detection algorithms. No automatic detection and measuring procedure or conceptual approach had ever been proposed or described to effectively and efficiently overcome these problems specific to detection and analysis of molecular combing data. For example, algorithms like those Zana or Lewis were not designed to solve these particular problems and each of them presents technical issues or impediments in the case of detection and measure of replication signals on molecular combing images. Consequently, the method of the invention is composite of several new and original elements that were designed, ordered and effectively combined to attain a full algorithmic model that solves the particular technical issues associated with analysis of DNA replication data obtained from molecular combing and similar laboratory procedures in a fast, robust and fully automated manner. The particular steps, the order of said steps, and the modification or adaptation of these steps each contribute to attaining the effective and efficient method of the invention.

In view of the above challenges, the inventors sought and have developed an efficient and accurate method for analysis of molecular combing image data describing DNA replication that is fast and robust and capable of precisely measuring DNA replication signals.

### BRIEF SUMMARY OF THE INVENTION

The computer-implemented algorithm of the invention automates the extraction of DNA replication data from images obtained from molecular combing. Different replication initiation and termination patterns are observed depending on when DNA replication begins or terminates. This method permits one to identify and detect at least seven different DNA replication patterns, including three replication initiation patterns, three replication termination patterns, and a unidirectional DNA replication pattern. Image data is obtained from replicating DNA that is differentially pulsed with two or more different labelled nucleotides. Unlabeled DNA can be counterstained to help identify longer DNA fibers containing both labeled and unlabeled segments.

Images of the stained and labeled DNA fibers are made and scanned.

These images are processed to remove noise and artifacts that are associated with molecular combing images and then analyzed for the presence of DNA initiation or termination patterns and other data obtained by molecular combing as described below.

The relevant connex components of the scanned images are extracted by normalized convolutions, adaptive thresholding, and mathematical morphological operations. An algorithm pour merges images of counterstained fibers or replicated nucleotide-labelled DNA segments that are sufficiently close thus eliminating sharpening the data by elimination of these artifacts. This algorithm is new and specific to linear components with molecular combing-related artifacts as described herein.

In addition to eliminating noise and sharpening molecular combing image data, the algorithm recognizes the seven different replication patterns described by the inventors and computes and extracts and analyzes relevant replication data from these patterns to calculate DNA replication speed, inter-origin distances, inter-termination distances, signal asymmetry and replication origin density.

The invention automates the identification and characterization of data obtained from molecular combing because it is not feasible to make these measurements or conduct analysis manually when large numbers of DNA replication patterns must be observed, characterized and quantified.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a DNA replication scheme involving a first (red) and second (green) pulse labelling of replicating DNA, using differentially labelled oligonucleotides iododeoxyuridine (iodo-dU; red) and chlorodeoxyuridine (chloro-dU; green). The location of the initiation of DNA replication as well as the time of initiation with respect to the time of pulse labelling with the two labelled oligonucleotides is determined from the DNA replication patterns. The process is shown as producing initiation 1 (I1) pattern on the left side of this figure.

FIG. 2 describes seven DNA replication signals. When two sequential pulses with different labelled nucleotides are performed these patterns are: a (I1) Initiations that start before first pulse labeling; (I2) Initiations that start during first pulse labeling; (I3) Initiations that start during second pulse labeling; (T1) Terminations that end during first pulse labeling; (T2) Terminations that end during second pulse labeling; (T3) Terminations that will end after second pulse labeling; and (U) Unidirectional signals (chains of red-green or green-red colored segments that do not belong to any of the initiations and terminations classes previously described). Other DNA replication signals or patterns are produced when only a single pulse, or when three, four or more pulses are performed. These other patterns may be deduced or projected from the patterns shown for two sequential pulses described in FIG. 2.

FIG. 3 is an image of DNA replication pattern Initiation 1 (I1). Replication of DNA started in the center of the blue segment in the middle of the pattern, continued when nucleotides labeled red were incorporated, and continued when nucleotides labeled green were incorporated on the ends of the I1 pattern.

FIG. 4 describes a global scheme of an RCA replication signal detection algorithm. Generally, molecular combing images coming from the scanner are very large (e.g., squares of 100 k pixel length) and these can be iteratively processed as sub-images (for RAM issues), from the channel splitting to the connex component extraction The fusion and the pattern detection may be performed once all sub-images have been processed. This global scheme comprises channel splitting, hue-based color filtering, extraction of connex components, merging of connex components, and pattern recognition as described in more detail below.

FIG. 5 describes a global scheme of the connex component extraction process.

FIGS. 6A and 6B. Compare speed distribution computed in an automatic manner or after a manual review of the signals for slide 1 (FIG. 6A) and slide 2 (FIG. 6B).

FIG. 7 provides examples of automatic detection of DNA replication signals. This figure includes DNA segments exhibiting U (green-red), I2 red-green-red), and I2 (red-green-red) patterns or signals. Vertical bars between opposed horizontal arrows indicate initiation points of DNA replication.

FIG. 8 provides examples of automatic detection of DNA replication signals. The boxes in this figure contain DNA segments exhibiting I1, T3, and I1 patterns or signals. Vertical bars indicate initiation points of DNA synthesis.

FIG. 9 illustrates a computer system upon which embodiments of the present disclosure may be implemented.

### DETAILED DESCRIPTION OF THE INVENTION

Molecular combing is a very efficient tool for analyzing information related to DNA replication. By consecutive pulse labeling with two thymidine analogs, iododeoxyuridine (IdU) and chlorodeoxyuridine (CldU), signals made from green and red segments are obtained. However, either one of these thymidine analogs or other known analogs may be used as a first or second pulse. The color of the first or second pulse label using tagged oligonucleotides is arbitrary. In the examples below the first pulse is red and the second pulse green. Labeling techniques using halogenated thymidine analogs are incorporated by reference to Yokochi, et al., Current Protocols in Cell Biology, Unit 22.10, DOI: 10.1002/0471143030.cb2210s35 (June, 2007).

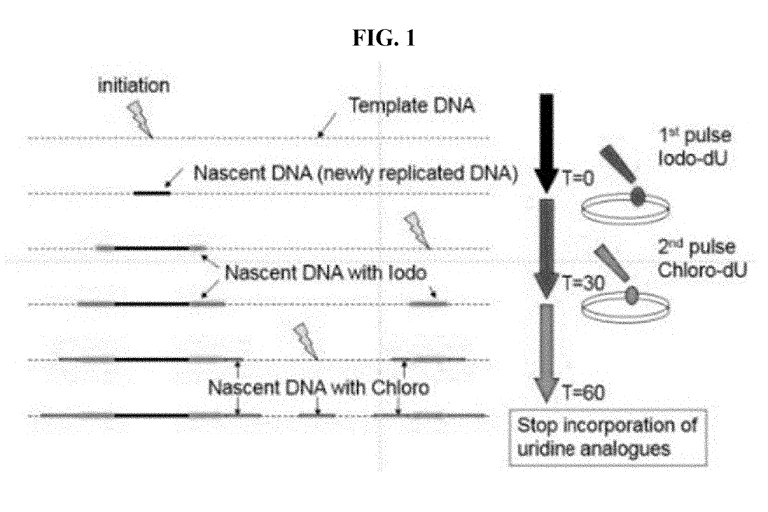

A scheme for differentially labeling replicating DNA is shown by FIG. 1. In order to understand if a fiber is broken or if two colored segments are belonging to the same fiber, a counterstaining (anti-ssDNA) is used during the revealing of DNA signals. The counterstained fibers appear in blue.

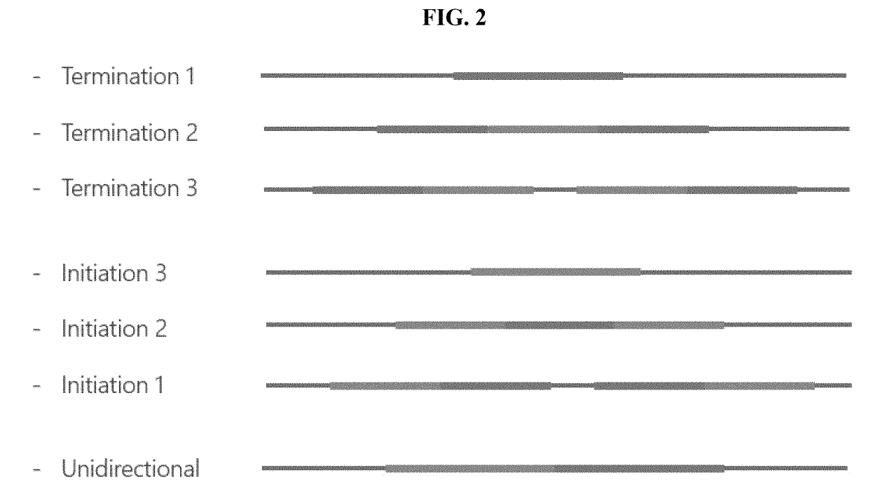

By pulse labeling with two different thymidine analogs with different labels (e.g., green or red), seven different classes of DNA replication patterns or signals were obtained. These constitute three initiation patterns (I1-I3), three termination patterns (T1-T3) and a unidirectional replication pattern (U). I1 initiations that start before the first pulse labeling, I2 initiations start during first pulse labeling and I3 initiations that start during second pulse labeling. Similarly, T1 terminations end during first pulse labeling, T2 terminations end during second pulse labeling, and T3 terminations that end after second pulse labeling. Unidirectional signals or patterns (“U”) are chains of red-green or green-red colored segments that do not belong to any of the initiations and terminations classes previously described. The seven classes of signals are shown by FIG. 2 where the red color corresponds to first pulse and the green color corresponds to the second pulse.

DNA replication patterns comprise a series of colored or multiple colored linear segments superimposed on long curvilinear fibers corresponding to DNA counterstaining.

The main difficulties for automating detection of DNA replication patterns or signals obtained by molecular combing arise from several sources: (i) the size of molecular combing signals can vary a lot, (ii) the images obtained with molecular combing contain numerous fluorescent artifacts (some of which are linear and can be confused with DNA replication patterns) and that can induce mistakes in the detection of DNA fibers and DNA replication signals, and (iii) quality of labeling can vary and result in partial fluorescence gaps in labeled DNA fibers and interrupted DNA replication patterns also known as sparking phenomena.

To address these problems, the inventors developed a computer-implemented method for (i) the automatic detection of DNA counterstained fibers and of the linear colored signals corresponding to replicated areas; (ii) the fusion of fibers and linear colored signals when there is a sparking phenomenon; and (iii) for the recognition of the different DNA replication patterns such as the I1, I2, I3, T1, T2, T3 and U patterns or signals described above.

Once the step of precise detection of fibers, segments and patterns is over, it is possible to estimate a set of information characterizing DNA replication, such as replication speed, inter-origin distances, inter-termination distances, signal asymmetry, and replication density.

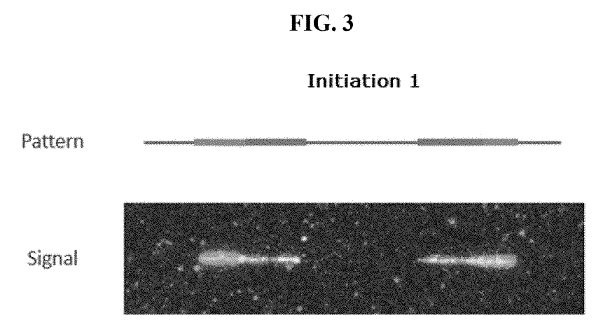

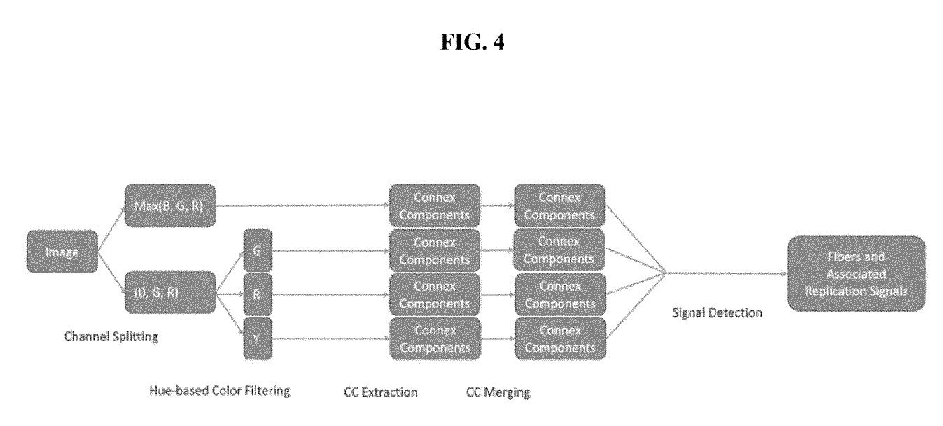

The images coming from the scanner are very large, for example, squares defined by pixel lengths of more than 50, 75, 100, 125, 150, 175, 200 k or similar quantities of data in other image forms or formats. The method disclosed herein permits such images to be accurately and efficiently processed and analyzed, for example to quantify the numbers of each kind DNA replication pattern or signal in an image in times of 5, 10, 15, 20, 30, 40, 50 or 60 minutes or less. FIG. 3 provide an example of a DNA replication pattern (T2) and background noise that has been identified and aligned with a T2 template using a method according to the invention.

The following disclosure describes the different steps of the computer-implemented algorithm of the invention, which is robust, fast and designed to deal with images that contain significant amounts of structured noise or where signal labeling is not uniform or optimal.

The present invention can be used for detecting spatial arrangements of nucleic acids, and specifically for studying DNA replication. By combining this process with a process for localizing one or more specific loci of the genome, it can be used for analyzing DNA replication characteristics on a set of specific genome loci.

The computer-implemented algorithm of the invention uses a combination of algorithmic steps and provides fast and robust detection of DNA replication patterns from molecular combining as well as precise measurement of the DNA replication patterns or signals. This algorithm comprises, but is not necessarily limited to the following steps. It may be performed in conjunction with molecular combing or be performed using molecular combing data that has been stored or transmitted.

The process consists of different sequential steps. First, the information corresponding to the replicated signals is separated from the information that corresponds to the counterstaining of DNA fibers (1). Afterwards, a splitting is performed using color information in order to separate the replication events corresponding to the first pulse labelling and those corresponding to the second pulse labelling (2). Then, some image processing techniques are applied for detecting the elements that have geometric properties that make them very likely to be parts of greater replication patterns (3). After this step, some elements may be separated but still very close and perfectly aligned, the gap between them can be caused by different artefacts in the image or by a lack of labelling intensity on some areas. Thus, a step of element merging is performed (4). Then, using all those detected elements, the patterns (chaining of colored elements and gaps between them) that build the DNA replication patterns of interest (5) are identified, quantified and characterized.

The method of the invention involves combinations of the following steps.

(1) Channel splitting. The first step of an image data processing algorithm concerns the separation of the information of the counterstaining and the information of replication signals. Counterstaining aims at highlighting DNA fibers, but its efficiency can be reduced on the replicated areas. Practically, replicated areas always correspond to DNA fibers. Thus, the following transformation is performed to obtain an image containing counterstaining information:
Image [B, G, R]→CS_image [max(B, G, R)]

Replication information is independent from counterstaining information. Thus, it was decided to perform the following transform to obtain an image containing replication-related information:

(2) Hue-based color filtering. The second step consists of obtaining gray-level images to separate replication information related to the first pulse labeling, replication information related to the second pulse labeling and artifact signals. To do that, a filter based on the Hue-Saturation-Value (HSV) representation of Replication_Image is used. The Hue component corresponds to the color and lets one easily separate the green and red colors that correspond to replicated areas, and the yellow potentially corresponding to artifact signals or mixed fibers. Three filters F_i=[hue_min_i; hue_max_i] for green, red and yellow are defined. By using each of those filters, the image corresponding to the Value channel where the Hue intensity is outside the interval defining the filter is masked. Four gray level images identified as “Gray_Level_Images” are obtained at this step: the one corresponding to counterstained areas, the one corresponding to areas replicated during the first pulse labeling, to areas replicated during the second pulse labeling and to areas corresponding to mixed fibers or artifacts.

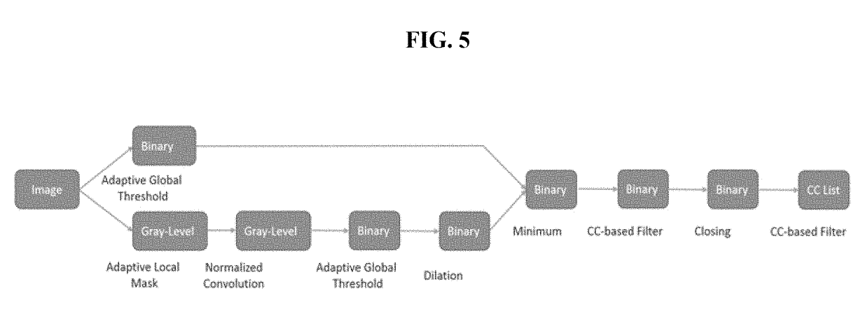

(3) Extraction of connex components. For each of those four images, a sequence of filtering operations and mathematical morphology operations in order to extract the set of relevant connex components in the images is conducted. These operations are described in this sub-section. The parameters used for the processing of fibers and the parameters used for the processing of replication signals are different, but the framework is the same. FIG. 5 shows a scheme of the connex component extraction process and depicts a global scheme of the connex component extraction process.

Two steps are performed in parallel on the gray-level images corresponding to each obtained channel. One step (containing adaptive local mask+normalized correlation+adaptive global threshold+dilation) aims at obtaining a mask that includes the areas of the image than potentially contain signals. However, because of potential side effects of the used operations, these regions are larger than the signal areas and thus do not follow exactly the contours of those signals. This part is used only for erasing areas that do not potentially contain signals. Because we are interesting in precise measurement of signals, we perform, in parallel, another step on the gray-level image (adaptive global thresholding) that lets us follow exactly the contours of the high-intensity areas of the image (that include the signals), and the minimum step that follows corresponds to erasing the areas that were not likely to contain signals from the obtained image. Afterwards, a filtering based on connex component areas+a closing operation+another filtering is performed for linking the components that may be separated because of a low quality staining and selecting the signals of sufficient size. Details of those steps are given in the following paragraphs:

i) Adaptive global threshold. Using the gray-level images Gray_Level_Image, an adaptive global thresholding in order to get rid of areas of insufficient intensity for being fibers or potential signals is performed. The computation of the threshold is performed in several steps. An aim is to identify the level that corresponds to the noise-level of the image for performing the thresholding above that level.

Signals that are close to horizontal are sought out by proceeding in the following manner: perform a 2D convolution with an horizontal rectangular window of integral 1 to compute the mean local values; perform a maximum operation on rows to obtain a profile corresponding to maximal mean local values of the different rows; and compute mean(profile)+A*standard_deviation (profile) and use the obtained value as a global threshold.

Local mean operation leads to a maximum operator that is robust to areas of high intensity but of size that is too small to be potential signals. It leads to a smoothing of the image without losing too much vertical resolution. The maximum operator leads to a ‘mean+A*standard-deviation’ operator that corresponds to a separation between the values of the rows corresponding to noise and those containing potential fibers or signals (even of small size) thus obtaining four binary globally thresholded images.

ii) Adaptive local mask. Using the gray-level images Gray_Level_Image, a local mask that aims at getting rid of areas that do not sufficiently come up locally or that are not of sufficient thickness is computed. In order to do that a 2D convolution operation is performed with a vertical rectangular window of integral 1 to compute mean local values Mean_Value_Image; perform an element-wise division of Gray_Level_Image by Mean_Value_Image to get a local contrast image Local_Contrast_image; perform filtering to get a binary image where Local_Contrast_Image is outside the interval [contrast_min, contrast_max] and use this binary image for masking gray-level images Gray_Level_Image, thus obtaining four gray level images identified as “Locally_Thresholded_Images”.

iii) Normalized convolution. Using the gray-level images identified as Locally_Thresholded_Images, a normalized convolution with a square filter with the third of the central rows contains 1 and 0 elsewhere was performed. This brings out areas that are approximately of the width of potential signals and of sufficient length thus providing four gray-level images identified as “Norm_Conv_Images”.

iv) Adaptive global threshold. Using the gray-level images identified as Norm_Conv_Images, an adaptive global threshold to identify the areas where relevant elements are present was performed. The computation of the threshold is performed in several steps. An aim is to identify a level that corresponds to the noise-level of the image for performing the thresholding above that level. Signals that are close to horizontal are sought by performing a maximum operation on row to obtain a profile corresponding to maximal values of the different rows; and compute mean(profile)+A*standard_deviation(profile) and using the obtained value as a global threshold. Unlike the global adaptive threshold performed in step i), the smoothing step of computing mean local values as the result of the convolution is not needed because the images are already smoothed at a scale that corresponding to the window size. This process provides four binary images identified as “Norm_Conv_Image_Bin”.

v) Dilation. Using binary images identified as Norm_Conv_Image_Bin, a dilation operation with an horizontal linear structural element of width 1 in order to spread the potential signal areas (useful when the signal width vary locally around the average signal width) was performed thus providing four binary images identified as “Norm_Conv_Dilated_Image_Bin”.

vi) Minimum. An element-wise minimum operation between binary images Norm_Conv_Dilated_Image_Bin and Globally_Thresholded_Image in order to detect in a precise manner areas of potential signals (relevant width and sufficient intensity) was performed to obtain four binary images identified as “Image_Bin”.

vii) Filter based on the areas of connex components. Using binary images Image_Bin, a detection of connex components and a filtering for getting rid of the components whose areas are inferior to some threshold was performed. This step de-noises the obtained binary images and provides four binary images identified as “Image_Bin_Denoised”.

viii) Closing. Using the binary images Image_Bin_Denoised, a morphological closing operation to link the signals or fibers when there is a sparking phenomenon was performed. This operation is a combination of a dilation with an horizontal rectangular structural element and of an erosion with an horizontal linear element of width 1. The difference between the width of the element used for dilation and the one used for erosion permits linkage of components when there are potentially oriented with a small angle with respect to horizontal thus providing four binary images identified as “Image_Bin_Denoized_Closed”.

ix) Filter using the areas of connex components. Using binary images Image_Bin_Denoized_Closed, a detection of connex components and a filtering for getting rid of the components whose areas are inferior to some threshold is performed thus providing four sets of connex components. Each connex component is represented by the set of coordinates of the pixels of the component (with coordinates relative to the global image).

(4) Merging of connex components. At this step, a fusion of aligned connex components is conducted. To do that, the set of couples of components that verify one or more alignment criteria is computed. Then, these components are fused in an iterative manner by linking them using parallelepiped polygons. The alignment criteria is defined as follows: Compute the right and left extremities of both connex components of interest (4 points) (see Algorithm 1, L3-4); For each distinct couple of points, compute the errors of a linear regression based on those two points (see Algorithm 1, L9) on the other two points (see Algorithm 1, L10) and sum those errors on all couples (see Algorithm 1, L11) and if this total error is inferior to some threshold and if the distance between neighboring central extremities of the components is inferior to some threshold (see Algorithm 1, L12-13), the criteria is verified. This new and original fusion scheme can then be written as the sequence of steps, such as, for example, the following steps:

Algorithm 1

L1 For each component C_1

| | |
|---|---|
| L2 | For each component C_2 |
| L3 | Compute extremity points of C_1 : P_1,left and P_1,right |
| L4 | Compute extremity points of C_2 : P_2,left and P_2,right |
| L5 | Store those 4 points in an array : Points_Array |
| L6 | Initialize the alignment error for this couple of elements : E_align = 0 |
| L7 | For each Point_i in Points_Array |
| L8 | For each Point_j in Points_Array |
| L9 | Compute the linear regression using Point_i and Point_j : LR |
| L10 | Compute the error E of LR on the other points |

Points_Array \ {Point_i, Point_j}

| | |
|---|---|
| L11 | Add the error E to E_align |
| L12 | If E_align < Threshold_1 and distance(extremities of elements) < Threshold_2 |
| L13 | Add the couple of elements to the list of components that need to be fused |
| | use the elements that need to be fused by inserting a parallelepiped |
| | element that links the right part of the left element with the left part of the |
| | right element for creating a fused larger element. |

Unlike previous methods, like those that use the Zana and Lewis imaging processing algorithms, the merging process according to the merges components that are aligned and close. The prior algorithms of Zana and Lewis only relate to image processing.

(5) Pattern recognition. Once all components have been fused, the set of patterns corresponding to the seven replication signals defined in section 1) is detected. In order to do that, the different fibers (connex components coming from the counterstaining) are run through. For each fiber, the set of replication-related connex components whose intersection with the considered fiber is superior to some threshold is computed. These components are sorted with respect to the horizontal axis and model the fiber as a string: 1 corresponding to components related to the first pulse, 2 corresponding to components related to the second pulse, 3 for those corresponding to mixed fibers and 0 for gaps (areas between 2 components and or size superior to some threshold). A search based on regular expressions is then used to find relevant candidate patterns. Finally, the candidates are filtered by adding constraints on minimal sizes of components or gaps.

Computer-Implementation.

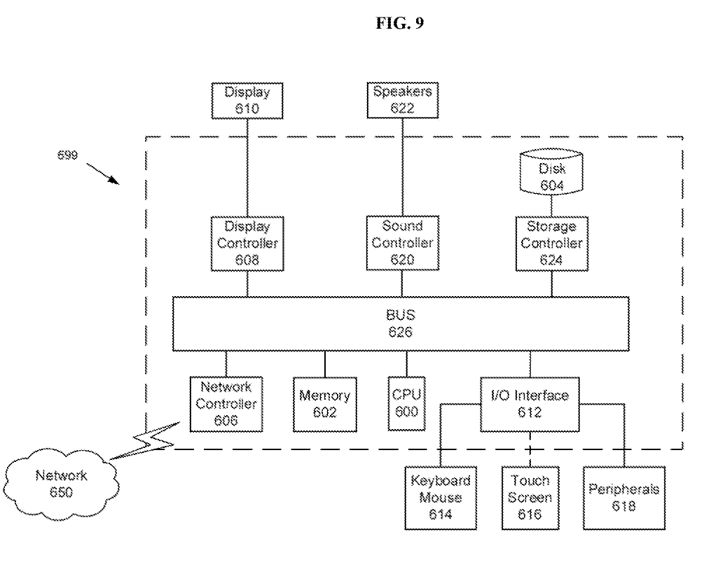

The algorithm disclosed herein is preferably transcribed into software and implemented on a computer. This permits analysis of quantities of molecular combing data that it would not be feasible to analyze manually. FIG. 9 illustrates a computer system upon which embodiments of the present disclosure may be implemented. Each of the functions of the above described embodiments may be implemented by circuitry, which includes one or more processing circuits. A processing circuit includes a particularly programmed processor, for example, processor (CPU) 600, as shown in FIG. 9. A processing circuit also includes devices such as an application specific integrated circuit (ASIC) and conventional circuit components arranged to perform the recited functions.

In FIG. 9, the device 699 includes a CPU 600 which performs the processes and implements the algorithms for analyzing molecular combing data described above. The device 699 may be a general-purpose computer or a particular, special-purpose machine. In one embodiment, the device 699 becomes a particular, special-purpose machine when the processor 600 is programmed to participate in processing and analyzing molecular combing data, and/or perform one or more steps of the process of FIG. 9.

The process data and instructions may be stored in memory 602. These processes and instructions may also be stored on a storage medium disk 604 such as a hard drive (HDD) or portable storage medium or may be stored remotely. The instructions may be stored on CDs, DVDs, in FLASH memory, RAM, ROM, PROM, EPROM, EEPROM, hard disk or any other device with which the system communicates, such as a server or computer. In other words, the instructions may be stored on any non-transitory computer-readable storage medium to be executed on a computer.

Further, the discussed embodiments may be provided as a utility application, background daemon, or component of an operating system, or combination thereof, executing in conjunction with CPU 600 and an operating system such as, but not limited to, Microsoft Windows, UNIX, Solaris, LINUX, Android, Apple MAC-OS, Apple iOS and other systems known to those skilled in the art.

CPU 600 may be any type of processor that would be recognized by one of ordinary skill in the art. For example, CPU 600 may be a Xenon or Core processor from Intel of America or an Opteron processor from AMD of America. CPU 600 may be a processor having ARM architecture or any other type of architecture. CPU 600 may be any processor found in a mobile device (for example, cellular/smart phones, tablets, personal digital assistants (PDAs), or the like). CPU 600 may also be any processor found in musical instruments (for example, a musical keyboard or the like).

Additionally or alternatively, the CPU 600 may be implemented on an FPGA, ASIC, PLD or using discrete logic circuits, as one of ordinary skill in the art would recognize. Further, CPU 600 may be implemented as multiple processors cooperatively working in parallel to perform the instructions of the processes described herein.

The computer 699 in FIG. 9 also includes a network controller 606, such as, but not limited to, a network interface card, for interfacing with network 650. As can be appreciated, the network 650 can be a public network, such as, but not limited to, the Internet, or a private network such as an LAN or WAN network, or any combination thereof and can also include PSTN or ISDN sub-networks. The network 650 can also be wired, such as an Ethernet network, or can be wireless such as a cellular network including EDGE, 3G and 4G wireless cellular systems. The wireless network can also be WiFi, Bluetooth, or any other wireless form of communication that is known.

The computer 699 further includes a display controller 608, such as, but not limited to, a graphics adaptor for interfacing with display 610, such as, but not limited to, an LCD monitor. A general purpose I/O interface 612 interfaces with a keyboard and/or mouse 614 as well as a touch screen panel 616 on or separate from display 610. General purpose I/O interface also connects to a variety of peripherals 618 including printers and scanners. The peripheral elements discussed herein may be embodied by the peripherals 618 in the exemplary embodiments.

A sound controller 620 may also be provided in the computer 699 to interface with speakers/microphone 622 thereby providing sounds and/or music. The speakers/microphone 622 can also be used to accept dictated words as commands.

The general purpose storage controller 624 connects the storage medium disk 604 with communication bus 626, which may be an ISA, EISA, VESA, PCI, or similar. A description of the general features and functionality of the display 610, keyboard and/or mouse 614, as well as the display controller 608, storage controller 624, network controller 606, sound controller 620, and general purpose I/O interface 612 is omitted herein for brevity as these features are known.

The automated method of the invention has several significant advantages over the currently used manual analysis of replication signals. First, the invention is able to process the image, detect and measure signals in the same time as the time of scan and a lot more precise and robust than manual analysis. At the same time, it can characterize various phenomena that occur very rarely in a cell, which would have been missed because of the smaller number of manually analyzed signals. As an example, it can lead to a precise quantification of blocked replication forks using an analysis of the asymmetry of the signals. Secondly, human selection and interpretation of signals can be subjective and human analysis is prone to different subject-specific biases or errors. With this innovative automated process, the parameters are fixed once and for all, and the results will be steady and comparable for any conditions at any time. Thirdly, the entire analysis for one image takes less than 40 minutes, which considerably accelerates the analysis and opens the path to very large-scale analysis of hundreds of different biological conditions or patients and millions of replication signals in practical time delays. Currently, this analysis is real-time as the time for scanning an image is about 1 hour. However, when new generation scanners are introduced in the future with a shorter scanning time, our method can still be greatly accelerated by the use of GPU optimized code or via a parallelization of the process on a network of linked computers on the cloud without any modification of the proposed method. The method of the invention is scalable, meaning that it is able to process images of greater size, from 50 k pixel length square images to IM or more pixel length square images in less than a few hours with parallelized and optimized implementation. The size of the image could be adapted to the frequency of the event we are trying to detect as the number of signals will be increasing along with the image size.

The following embodiments, which are non-limiting, describe various aspects of the present invention. They are not to be construed to limit the claims in any manner whatsoever.

1. A computer-implemented method for detecting and analyzing DNA replication patterns, comprising:

a) incubating DNA undergoing replication in the presence of a first labeled nucleotide under conditions where the first labeled nucleotide is incorporated into replicating DNA and produces a first color-coded signal, and then

b) incubating the DNA in the presence of a second labeled nucleotide different from the first labeled nucleotide under conditions where the first labeled nucleotide is incorporated into replicating DNA and produces a second color-coded signal different than the color-coded signal of the first labeled nucleotide;

c) counterstaining unlabeled DNA in a third color distinguishable from those of the first and second labeled nucleotides;

d) stretching or otherwise aligning the DNA from step c) on a substrate;

e) acquiring an image of the stretched or otherwise aligned DNA,

f) analyzing the image using a computer-implemented algorithm, and

g) displaying or outputting a result of the analysis;
wherein the DNA replication patterns are selected from the group consisting of initiation signal 1, initiation signal 2, initiation signal 3, termination signal 1, termination signal 2, termination signal 3, and a unidirectional replication signal, or combinations thereof; and
wherein the analyzing quantitatively detects the numbers of one or more DNA replication patterns and/or lengths of one or more DNA replication patterns formed by the two labeled nucleotides incorporated into the DNA during replication.

In this embodiment and related embodiments, each color-coded signal is distinguishable from the others, for example, as described embodiments 2 and 3. Counterstaining serves to identify DNA fibers or filaments and may be performed using a blue or other color counterstain that can be distinguished from segments labeled with the first or second labels.

2. The method according to embodiment 1 where the three different colors are red, green and blue.

3. The method according to embodiment 1 or 2 where the first color-coded signal is red, the second color-coded signal is green, and the third counterstain color is blue.

In some expanded embodiments, additional color-coded nucleotides may be used, for example, in step b to label replicating DNA, such as a third or fourth color-coded nucleotide which are distinguishable from other color-coded nucleotides. Alternatively, the method may be performed using a pattern of pulses of different color-coded nucleotides, such as an alternation of nucleotides encoded green and red or green and red.

In some embodiments, the DNA further may be contacted with a mapping probe that binds to a predetermined genetic locus to provide a referent for mapping the replicating DNA sequence being analyzed. These embodiments involve evaluating the replication activity of a given locus, the combed DNA molecules are hybridized with a combination of complementary fluorescent probes, designed to specifically recognize one or more regions of interest. As a result, the DNA sequence to be analyzed is labelled with a succession of “dashes and dots”, creating a Genomic Morse Code (GMC) specific to the targeted gene or polynucleotide sequence and the flanking regions. Classically, several colors and/or probes of varying lengths can be used to cover a large genomic region as described by Cheeseman, K.; Rouleau. E.; Vannier, A.; Thomas, A.; Briaux, B.; Lefol, C.; Walrafen, P.; Bensimon, A.; Lidereau, R.; Conseiller, E.; Ceppi, M.; Human Mutation. 33(6):998-1009, 2012 (incorporated by reference). In some cases these strategies are not suitable owing to spectral overlap with the labeling of thymidine analogs used in RCA signals and non-specific hybridization of repetitive sequences, respectively. However, gaps of different sizes, defined by unicolor and similar size probes can provide the same information as probes of different color or size; see Lebofsky, R.; Heiling, R.; Sonnleitner, M.; Weissenbach, J.; Bensimon, A.; Molecular Biology of the Cell. 17(12):5337-45, 2006 (incorporated by reference)). Thus, DNA molecules can still be oriented even though the complete set of probes is not visualized. As gaps provide positional information, their numbers are no longer limited and spectral overlap and the presence of repetitive elements are not significant problems.

Alternatively, in other embodiments, several unicolor probes of different sizes can be used to identify large genomic regions up to several hundred of kilobases. BAC clones partly covering the region of interest conventionally serve as matrices for the synthesis of the probes by random priming; Stevanoni, M.; Palumbo, E.; Russo, A.; PLoS Genetics. 12(7):e1006201.doi: 10.1371/journal.pgen.1006201.2016 (incorporated by reference).

Images of DNA replication patterns in the embodiments described herein may be of different shapes and sizes. Image data obtained from molecular combing may be of various sizes and is usually too extensive to be practical to manually analyze. Images may be rectangular or square and comprise pixel lengths of 50,000, 100,000, or 200,000 pixels or more on each side as described by embodiments 4, 5 and 6.

An image is represented as a two dimensional array of pixels, where each pixel can have several values that correspond to different colors. Such an image depicts macromolecules containing colored patterns of replicated signals as described above.

An image is composed of stitched sub-images where each sub-image corresponds to the scanner's field of view.

Each field of view be may be scanned with several fluorophores where each fluorophore will be associated with a respective color. For example, if three fluorophores are used and associated with colors, e.g., red, green and blue, then each pixel will have three values. The size of the sub-image is typically 2,000×2,000 pixels and horizontal and vertical number of sub-images are typically 50×50. All the sub-images stitched together represent one color image of size 100,000×100,000 pixels.

4. The method of embodiments 1, 2, or 3, wherein the images are rectangular or square and comprise pixel lengths of 50,000 pixels or more on each side.

5. The method of embodiments 1, 2, 3 or 4, wherein the images are rectangular or square and comprise pixel lengths of 100,000 pixels or more on each side.

6. The method of embodiments 1 to 5 in particular of 1, 2, or 3, wherein the images are rectangular or square and comprise pixel lengths of 200,000 pixels or more on each side.

The methods described herein, such as those of embodiments 1-6 above may be performed in order to identify and analyze one or more different types of DNA replication patterns, including the seven different replication patterns described herein as described by embodiment 7.

7. The method of embodiments 1 to 6 in particular of 1, 2, or 3, wherein the analyzing detects the numbers of one or more DNA replication patterns of the same type.

The methods of the invention, such as those of embodiments 1-7 above, may also be used to identify and statistically characterize features of a particular type of DNA replication pattern, such as DNA length, as described by embodiment 8 below.

8. The method of embodiment 1 to 7, wherein the analyzing determines the minimum, maximum and average length and standard deviation of one or more DNA patterns of the same type.

The methods of the invention, such as those of embodiments 1-8 above, advantageously distinguish between unlabeled DNA which is counterstained and replicating DNA labeled with a first or second color-coded nucleotide. They also may distinguish between a true DNA replication pattern and artifact patterns with gaps, more than one color (often yellow), or overlapping colors. Such embodiments, including specific embodiments 9 through 12, may employ a channel-splitting algorithm or use filters, such as hue-based filters, to identify and exclude artifacts.

9. The method of any one of embodiments 1 to 8 in particular of 1, 2, or 3, wherein the algorithm (i) distinguishes between unlabeled counter-stained DNA and DNA labeled with the first labeled nucleotide and the second labeled nucleotide and (ii) distinguishes between DNA incorporating the red first labeled nucleotide, the green second labeled nucleotide, and yellow artifacts or mixed fibers.

10. The method of any one of embodiments 1 to 9 in particular of 1, 2, or 3, wherein the algorithm comprises a channel-splitting algorithm that distinguishes between unlabeled counter-stained DNA and DNA labeled with the first labeled nucleotide and the second labeled nucleotide.

11. The method of any one of embodiments 1 to 10 in particular of 1, 2, 3, wherein the algorithm comprises hue-based color filtering and distinguishes between DNA incorporating the red first labeled nucleotide, the green second labeled nucleotide, and yellow artifacts or mixed fibers.

12. The method of embodiment 11, wherein the image is filtered through three hue-based color filters for green, red and yellow thus providing four gray level images respectively depicting counterstained segments, areas replicated during a first pulse with the first labeled nucleotide, areas replicated during a second pulse with the second labeled nucleotide, and areas corresponding to mixed fibers and artifacts.

In some embodiments, such as embodiments 13 and 14, the method as described herein and by preceding specific embodiments will quantitatively detect and display numbers of one or more DNA replication patterns detected.

13. The method of any one of embodiments 1 to 13, in particular 1, 2, 3 that comprises quantitatively detecting and displaying the number of a particular DNA replication pattern in the image, or the respective numbers of two or more particular DNA replication patterns.

14. The method of any one of embodiments 1 in particular of 1, 2, or 3 that comprises quantitatively detecting the number of a particular DNA replication pattern in the image and at least one of a length range of said DNA replication pattern in the image, an average length of said DNA replication pattern in the image, or the standard deviation of length ranges of the DNA replication patterns of the same type in the image.

Some embodiments of these methods will involve processing image data obtained from at least 2, 3, 4, or more DNA samples taken from different individuals, different tissues in an individual (such as normal or control tissue and from cancerous tissue or pharmaceutically, radiologically or genetically treated tissue), or tissues from the same individual recovered at different times such as over the course of a disease or treatment.

The image data from multiple or compared samples may be processed to identify or characterize one or more different DNA replication patterns. For example, the method can be performed on two or more DNA samples in order to compare replication parameters or map between or among the different DNAs. Thus, it can identify the replication patterns of each sample, detect at least one spatial difference between DNA replication patterns of a control DNA and test DNA, detect at least one temporal difference between DNA replication patterns of a control DNA and test DNA, distances between origins of replication in the control and test replicating DNA molecules, detect replication speed differences between the control and test DNA replication patterns, detect differences in inter-termination distances between the control and test replicating DNA molecules, detect differences in DNA signal symmetry between the control and test replicating DNA molecules, detect differences in density of the origins of replication between the control and test replicating DNA molecules, detect differences in eye-to-eye distances between the control and test replicating DNA molecules, detect differences in size distributions of eyes between the control and test replicating DNA molecules.

Other DNA replication parameters may also be analyzed as long as they can be represented by the DNA patterns described herein or patterns derived therefrom. These include, but are not limited to those described by Techer, et al., J. Mol. Biol. 425: 4845-4855 (2013).

In some embodiments, the method disclosed herein can quantitatively detect and display the number of a particular DNA replication pattern in the image, or the respective numbers of two or more particular DNA replication patterns; quantitatively detect the number of a particular DNA replication pattern in the image and at least one of a length range of said DNA replication pattern in the image, an average length of said DNA replication pattern in the image, or the standard deviation of length ranges of the DNA replication patterns of the same type in the image. These aspects of the invention include but are not limited to embodiments 15-24.

15. The method of any one of embodiments 1 to 14 in particular of 1, 2, or 3 that is performed on at least two different DNA samples and that comprises comparing the replication patterns of said samples.

16. The method of embodiment 15 that comprises detecting at least one spatial difference between DNA replication patterns of a control DNA and test DNA.

17. The method of embodiment 15 that comprises detecting at least one temporal difference between DNA replication patterns of a control DNA and test DNA.

18. The method of embodiment 15 that comprises detecting distances between origins of replication in the control and test replicating DNA molecules.

19. The method of embodiment 15 that comprises detecting replication speed differences between the control and test DNA replication patterns.

20. The method of embodiment 15 that comprises detecting differences in inter-termination distances between the control and test replicating DNA molecules.

21. The method of embodiment 15 that comprises detecting differences in DNA signal symmetry between the control and test replicating DNA molecules.

22. The method of embodiment 15 that comprises detecting differences in density of the origins of replication between the control and test replicating DNA molecules.

23. The method of embodiment 15 that comprises detecting differences in eye-to-eye distances between the control and test replicating DNA molecules.

24. The method of embodiment 15 that comprises detecting differences in size distributions of eyes between the control and test replicating DNA molecules.

Other embodiments of the invention include but are not limited to methods for detecting or evaluating changes or variations in DNA replication, replication activity, positioning, or for standardizing, monitoring, and verifying reconstruction of one or more genetic modifications, such as those described by embodiments 25-27, 28, 29 or 30 and 31. Like the other methods described herein, these methods are not limited to detection of variations in human DNA, but may be performed on DNA from mammals or other eukaryotes, or on that of prokaryotes, especially those organisms or tissues which have been mutated, transformed, genetically engineered, epigenetically altered, or have had their genomes otherwise modified. Such methods may also be performed on DNA obtained from tissue ex vivo or in vitro, such as a stored or cultured cell line or tissue. Such a method may be used for detecting variations in DNA replication comprising comparing control DNA replication patterns of a normal or unmodified cells with test DNA replication patterns of genetically-modified cells. For example, it may be used to compare or evaluate DNA replication patterns from a cell that has been genetically modified 2016). For example, by using the system CRSPR (see e.g., Le Cong, et al., Science 339(6121):819-823 (2013, incorporated by reference) or the cells obtained from a subject who has undergone a therapeutic genetic modification, the present invention allows one to measure the level of the DNA replication and/or the location of replication patterns after such modifications. However, DNA samples may be obtained, analyzed and compared from any source in which replicating DNA occurs, including animals, plants and microorganisms, for example, to detect biological variability between organisms or between organisms at different stages of development.

25. A method for detecting variations in DNA replication comprising comparing control DNA replication patterns of a normal or unmodified cells with test DNA replication patterns of genetically-modified cells, wherein said method comprises the method of embodiment 1 to 24.

26. The method of embodiment 25, wherein the test DNA replication patterns are obtained from a cell that has been genetically modified using CRSPR.

27. The method of embodiment 25 or 26, wherein the test DNA replication patterns are obtained from cells of a subject who has undergone a therapeutic genetic modification.

Another embodiment of the method is directed to determining the position of a genetic locus, gene, or other polynucleotide, which is present in more than one copy in a genome, comprising comparing DNA replication patterns of a normal or unmodified cells with test DNA replication patterns of genetically-modified cells and optionally mapping the DNA, for example, by the use of probes to known genes or polynucleotide sequences. This aspect of the invention includes, but is not limited to that of embodiment 28.

28. A method for determining the position of a gene or other polynucleotide, which is present in more than one copy in a genome, comprising comparing DNA replication patterns of a normal or unmodified cells with test DNA replication patterns of genetically-modified cells, wherein said method comprises the method of any one of embodiments 1 to 24 in particular of 1, 2, or 3.

29. A method for evaluating replication activity in eukaryotic cells comprising:
a. preparing labelled and purified DNA,
b. stretching or combing the DNA of (a),
c. visualizing and analyzing replication activity of the DNA according to the method of any one of embodiments 1 to 24 in particular of 1, 2, or 3;
d. optionally, comparing the analysis from (c) to a reference normal cell; and
e. optionally, quantifying the replication activity in the tested cells and the reference normal cells.

In some embodiments, the method disclosed herein may be used for standardizing, monitoring, and verifying the reconstruction of one or more genetic modifications that have occurred in or been induced into a genome. The method of the invention may be employed for analyzing the images of DNA replication events for the evaluation of the DNA replication activity in various circumstances, such as the effect of a drug or drug combination on the nature, occurrence or timing of DNA replication as well as the effects of DNA replication inhibitors such as cisplatin/carboplatin, which can perturb progress of replication forks, and cdc7 inhibitors, which can interfere with initiation of DNA replication. The method may also be used for identifying the presence of absence of a genetic defect in a DNA replication pathway or used in conjunction with gene modification or gene therapy to assess correction of genetic defects, for example, by comparison of DNA replication data for unmodified, modified and control cells, such as embodiments 30 and 31 below.

30. A method for standardizing, monitoring, and verifying reconstruction of one or more genetic modifications that have occurred in or been induced into a genome comprising detecting and analyzing DNA replication patterns of at least one genetically-modified DNA sequence according to any one of embodiments 1 to 24 in particular of 1, 2, or 3.

31. The method of embodiment 30, further comprising comparing DNA replication patterns of the at least one genetically-modified DNA sequence with those of an unmodified DNA sequence or control sequence.

Yet another embodiment of the invention pertains to a machine-readable storage medium, (such as a computer readable storage medium), comprising a program to execute procedures for molecular combing or for analyzing image data from molecular combing or similar procedures. The machine readable storage medium may comprise a program containing a set of instructions to execute procedures for detecting polynucleotides that incorporate nucleotides tagged with at least one of two different colored or other detectable labels comprising: (a) imaging or scanning a surface containing polynucleotides incorporating said nucleotides to obtain luminescent or other detectable signals from said polynucleotides; (b) converting the luminescent signals or other detectable signals into digital data; (c) utilizing the digital data to automatically measure integrity and length of the polynucleotides incorporating tagged nucleotides, the integrity and length of segments labeled with each of the two colored nucleotides, and the one or two color labeling patterns exhibited by each labeled polynucleotide, wherein a labeling pattern of the polynucleotide indicates the initiation or termination of polynucleotide replication of the polynucleotide; and (d) displaying results of labeling patterns of the at least one labeled polynucleotide to a user.

A machine readable storage medium includes, but is not limited to magnetic disks, cards, tapes, drums, flash drives, hard disks, floppy disks, optical discs, barcodes, and magnetic ink characters or any other medium that is capable of storing image data in a format readable by a mechanical device in contrast to a medium readable by a human.

The machine readable storage medium may contain instructions for identification and characterization polynucleotide replication patterns (such as patterns 1-7 as disclosed herein) and with instructions for standardizing, monitoring, and verifying the reconstruction of one or more genetic modifications that have occurred in or been induced into a genome or other polynucleotide.

This machine readable storage medium may further comprise (a) imaging or scanning a surface containing polynucleotides incorporating said nucleotides to obtain luminescent signals from said polynucleotides; wherein the polynucleotide is further counterstained to obtain visual signals from segments that did not incorporate the tagged nucleotides; or may further comprise (a) imaging or scanning a surface containing polynucleotides incorporating said nucleotides to obtain luminescent signals or other detectable signals from said polynucleotides; wherein, optionally, the polynucleotide is further contacted with one or more labeled marker probes that bind to predefined sequences in a polynucleotide and obtaining visual signals from said bound labeled marker probes. This aspect of the invention includes but is not limited to those described by embodiments 32-41.

32. A machine readable storage medium comprising a program containing a set of instructions to execute procedures comprising:

a) imaging or scanning a surface containing polynucleotides incorporating nucleotides tagged with at least one of two detectable labels to obtain detectable signals from said polynucleotides;

b) converting the detectable signals into digital data;

c) utilizing the digital data to determine integrity and length of the polynucleotides incorporating tagged nucleotides, the integrity and length of each detectably labeled segment of the polynucleotides, and one or more patterns of detectably labeled segments in the polynucleotides, wherein a labeling pattern exhibited by a polynucleotide indicates a position of DNA initiation or termination in the polynucleotide; and

d) displaying results of labeling patterns exhibited by the at least one labeled polynucleotide to a user.

33. The machine readable storage medium of embodiment 32 that comprises instructions for scanning a surface containing polynucleotides incorporating at least one of two different color-coded nucleotides to obtain luminescent signals from said polynucleotides.

34. The machine readable storage medium of embodiment 32 that comprises instructions for scanning a surface that is rectangular or square and that comprises pixel lengths of 50,000 pixels or more on each side.

35. The machine readable storage medium of embodiment 32 that comprises instructions for scanning a surface that is rectangular or square and that comprises pixel lengths of 100,000 pixels or more on each side.

36. The machine readable storage medium of embodiment 32 that comprises instructions for scanning a surface that is rectangular or square and that comprises pixel lengths of 200,000 pixels or more on each side.

37. The machine readable storage medium of embodiment 32 that comprises instructions for (a) scanning a surface containing polynucleotides incorporating said nucleotides to obtain detectable signals from said polynucleotides; wherein the polynucleotide is further counterstained or tagged to identify segments that did not incorporate the tagged nucleotides.

38. The machine readable storage medium of embodiment 32 that comprises (a) scanning a surface containing polynucleotides incorporating said detectable nucleotides to obtain signals from said polynucleotides; wherein the polynucleotide is further contacted with one or more detectable marker probes that bind to predefined sequences in a polynucleotide and detecting signals from said bound detectable marker probes.

39. A method for detecting, characterizing or analyzing DNA replication patterns comprising using the machine readable storage medium of embodiment 32 to receive, process and analyze signals from detectably labeled polynucleotides and counterstained unlabeled nucleic acids.

Some embodiments of the invention will involve detection and analysis of DNA replication patterns associated with an infectious, immunological, or genetic disease and to longitudinal analysis of DNA replication in patients undergoing or who have undergone therapeutic or corrective gene therapy. Patients include humans, mammals, avians, fish and other commercially valuable species as well as assessment of DNA replication patterns in transgenic animals, for example as compared to non-transgenic animals.

Genetic modifications include those associated with treatment of genetic disorders such as severe combined immunodeficiency and Leber's congenital amaurosis, or treatments for cystic fibrosis, sickle cell anemia, Parkinson's disease, cancer, diabetes, color-blindness, heart disease, muscular dystrophy or other genetic disorders.

Such methods may also be used to study DNA replication patterns in virus-infected cells, such as those infected with retroviruses like HIV or hepatitis viruses such as HAV, HBV or HCV or in cells invaded by other microorganisms such as mycobacteria, rickettsia, chlamydia, or plasmodium. Such analysis may also be conducted before and after a treatment (such as a radiological, surgical, or pharmacological treatment including but not limited to treatment with antiviral, antimicrobial, or anticancer drugs, or with immunopotentiators, immunomodulators, or immunosuppresants) for a particular disease, disorder or condition.

Similarly DNA replication patterns associated with sensory limitations such as hearing loss, olfactory or gustatory loss, tactile limitations, balance, alopecia, or other phenomena often associated with aging or exposure to drugs or toxins may be analyzed. DNA replication patterns associated with other disorders or conditions, such as obesity, weight loss, or exposure to a zero-g or weightless environment may be analyzed. Similarly DNA replication patterns associated with psychological or psychiatric conditions may be analyzed. Embodiments 40-41 describe non-limiting embodiments of this aspect of this technology.

40. A method for standardizing, monitoring, and verifying reconstruction of one or more genetic modifications that have occurred in or been induced into a genome comprising detecting and analyzing DNA replication patterns of at least one genetically-modified DNA sequence according to embodiment 1 to 24.

41. The method of embodiment 40, further comprising comparing DNA replication patterns of the at least one genetically-modified DNA sequence with those of an unmodified DNA sequence or control sequence.

### Example 1

### Comparison of Manual and Automated Processing of Molecular Combing Data

Imaged molecular combing data from two separate slides was used to estimate DNA replication speed by automated and manual analysis. The slides contained a limited amount of data; however, each contained a sufficient amount of molecular combing data to provide a useful comparison manual and automated processing.

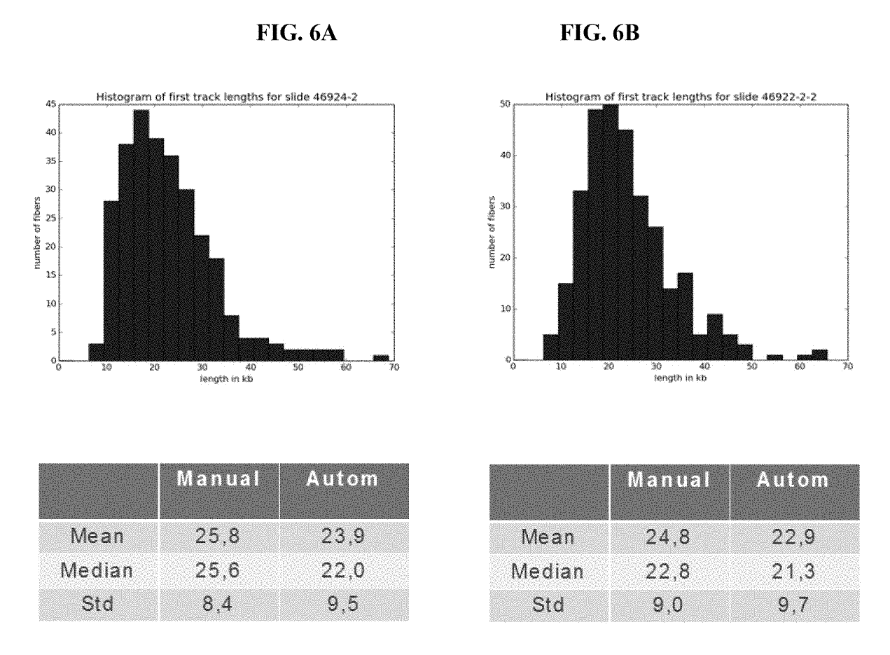

One of the main parameters of DNA replication is replication speed. This can be estimated in a global manner based on the size of internal colored segments in the replication patterns, such as the internal segments of replication pattern I1. The replication patterns imaged on each slide were analyzed to determine DNA replication speed and the results are shown in FIGS. 6A and 6B. As apparent from these figures, the results of automated and manual data analysis were close indicating that accuracy of automated analysis was similar to that of manual analysis.

### Example 2

### Identification of DNA Replication Patterns Using Computer-Implemented Algorithm

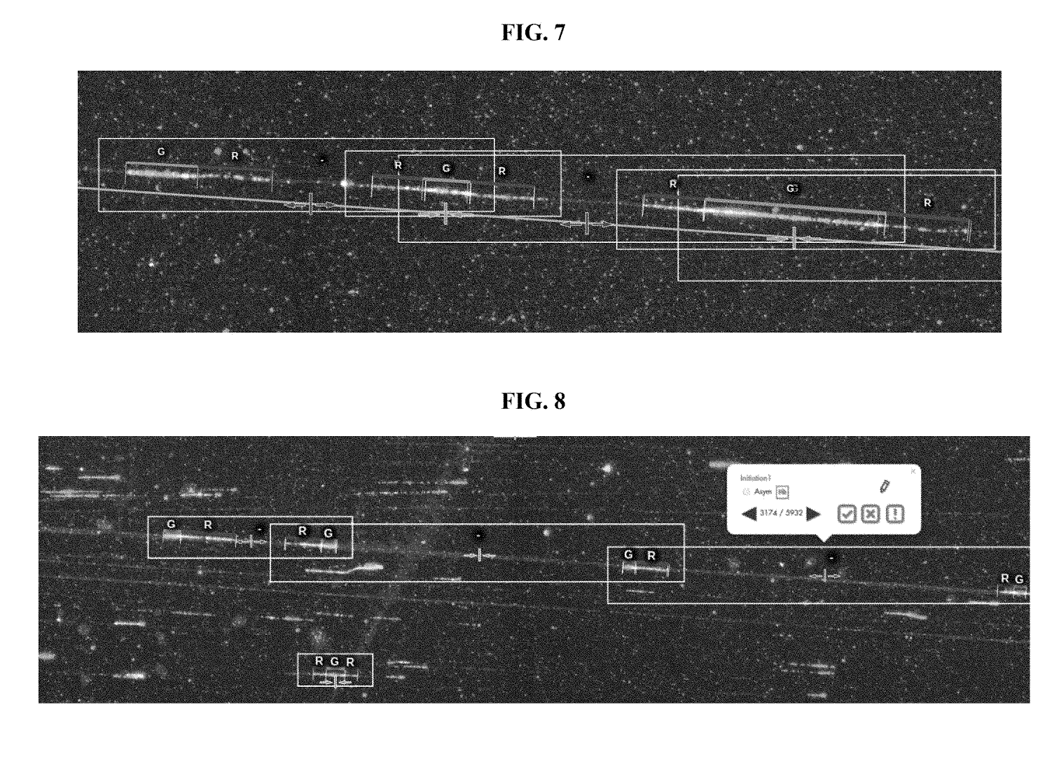

Image data from molecular combing was processed using the computer-implemented method described herein. DNA replication patterns were identified as shown by FIGS. 3, 7 and 8. Some sub-signals (color segments) can be part of several signals. Two close initiation firings end forming a termination. As shown in FIG. 8 the signals that have not been efficiently counterstained have not been detected. This figure presents results of a parametrization of the algorithm that aims at detecting patterns that are substantially entirely counterstained (before and after the signals as well as underlying them).

Terminology used herein is for the purpose of describing particular embodiments only and is not intended to be limiting of the invention.

The headings (such as “Background” and “Summary”) and sub-headings used herein are intended only for general organization of topics within the present invention, and are not intended to limit the disclosure of the present invention or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

As used herein, the singular forms “a”, “an” and “the” are intended to include the plural forms as well, unless the context clearly indicates otherwise.

As used herein, the term “and/or” includes any and all combinations of one or more of the associated listed items and may be abbreviated as “/”.

Links are disabled by insertion of a space or underlined space before “www” and may be reactivated by removal of the space.

As used herein in the specification and claims, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc. Any numerical range recited herein is intended to include all sub-ranges subsumed therein.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology. As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears. The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

1. A computer-implemented method for detecting and analyzing DNA replication patterns, comprising:
a) incubating DNA undergoing replication in the presence of a first labeled nucleotide under conditions where the first labeled nucleotide is incorporated into replicating DNA and produces a first color-coded signal, and then
b) incubating the DNA in the presence of a second labeled nucleotide different from the first labeled nucleotide under conditions where the first labeled nucleotide is incorporated into replicating DNA and produces a second color-coded signal different than the color-coded signal of the first labeled nucleotide;
c) counterstaining DNA in a third color distinguishable from those of the first and second labeled nucleotides;
d) stretching or otherwise aligning the DNA from step c) on a substrate;
e) acquiring an image of the stretched or otherwise aligned DNA,
f) analyzing the image using a computer-implemented algorithm, and
g) displaying or outputting a result of the analysis;
wherein the analyzing quantitatively detects the numbers of one or more DNA replication patterns and/or lengths of one or more DNA replication patterns formed by the two labeled nucleotides incorporated into the DNA during replication, and
wherein the DNA replication patterns are selected from the group consisting of initiation signal 1, initiation signal 2, initiation signal 3, termination signal 1, termination signal 2, termination signal 3, and a unidirectional replication signal, or combinations thereof.

2. The method according to claim 1 where the three different colors are red, green and blue.

3. The method according to claim 1 where the first color-coded signal is red, the second color-coded signal is green, and the third counterstain color is blue.

4. The method of claim 1, wherein the images are rectangular or square and comprise pixel lengths of 100,000 pixels or more on each side.

5. The method of claim 1, wherein the analyzing detects the numbers of one or more DNA replication patterns of the same type.

6. The method of claim 1, wherein the analyzing determines the minimum, maximum and average length and standard deviation of one or more DNA patterns of the same type.

7. The method of claim 1, wherein the algorithm (i) distinguishes between unlabeled counter-stained DNA and DNA labeled with the first labeled nucleotide and the second labeled nucleotide and (ii) distinguishes between DNA incorporating the red first labeled nucleotide, the green second labeled nucleotide, and yellow artifacts or mixed fibers.

8. The method of claim 1, wherein the algorithm comprises a channel-splitting algorithm that distinguishes between unlabeled counter-stained DNA and DNA labeled with the first labeled nucleotide and the second labeled nucleotide.

9. The method of claim 1 that comprises quantitatively detecting and displaying the number of a particular DNA replication pattern in the image, or the respective numbers of two or more particular DNA replication patterns.

10. The method of claim 1 that comprises quantitatively detecting the number of a particular DNA replication pattern in the image and at least one of a length range of said DNA replication pattern in the image, an average length of said DNA replication pattern in the image, or the standard deviation of length ranges of the DNA replication patterns of the same type in the image.

11. The method of claim 1 that is performed on at least two different DNA samples, a control sample and a test sample, and that comprises comparing the replication patterns of said samples.

12. The method of claim 11 that comprises detecting distances between origins of replication in the control and test replicating DNA molecules.

13. The method of claim 11 that comprises detecting replication speed differences between the control and test DNA replication patterns.

14. The method of claim 11 that comprises detecting differences in inter-termination distances between the control and test replicating DNA molecules.

15. The method of claim 11 that comprises detecting differences in size distributions of eyes between the control and test replicating DNA molecules.

16. A method for detecting variations in DNA replication comprising comparing control DNA replication patterns of normal or unmodified cells with test DNA replication patterns of genetically-modified cells, wherein said method comprises the method of claim 1.

17. The method of claim 16, wherein the test DNA replication patterns are obtained from a cell that has been genetically modified using CRSPR.

18. The method of claim 16, wherein the test DNA replication patterns are obtained from cells of a subject who has undergone a therapeutic genetic modification.

19. A method for determining the position of a gene or other polynucleotide, which is present in more than one copy in a genome, comprising comparing DNA replication patterns of normal or unmodified cells with test DNA replication patterns of genetically-modified cells, wherein said method comprises the method of claim 1.

20. A method for evaluating replication activity in eukaryotic cells comprising:
a. preparing labelled and purified DNA,
b. stretching or combing the DNA of (a),
c. visualizing and analyzing replication activity of the DNA according to the method of claim 1;
d. optionally, comparing the analysis from (c) to that of a reference normal cell; and
e. optionally, quantifying the replication activity in the tested cells and the reference normal cells.

21. A method for standardizing, monitoring, and verifying reconstruction of one or more genetic modifications that have occurred in or been induced into a genome comprising detecting and analyzing DNA replication patterns of at least one genetically-modified DNA sequence according to claim 1.

22. The method of claim 21, further comprising comparing DNA replication patterns of the at least one genetically-modified DNA sequence with those of an unmodified DNA sequence or control sequence.

23. A machine readable storage medium comprising a program containing a set of instructions to execute procedures comprising:
a) imaging or scanning a surface containing polynucleotides incorporating nucleotides tagged with at least one of two detectable labels to obtain detectable signals from said polynucleotides;
b) converting the detectable signals into digital data;
c) utilizing the digital data to determine integrity and length of the polynucleotides incorporating tagged nucleotides, the integrity and length of each detectably labeled segment of the polynucleotides, and one or more patterns of detectably labeled segments in the polynucleotides, wherein a labeling pattern exhibited by a polynucleotide indicates a position of DNA initiation or termination in the polynucleotide; and
d) displaying results of labeling patterns exhibited by the at least one labeled polynucleotide to a user.

24. The machine readable storage medium of claim 23 that comprises instructions for scanning a surface containing polynucleotides incorporating at least one of two different color-coded nucleotides to obtain luminescent signals from said polynucleotides.

25. The machine readable storage medium of claim 23 that comprises instructions for (a) scanning a surface containing polynucleotides incorporating said nucleotides to obtain detectable signals from said polynucleotides; wherein the polynucleotide is further counterstained or tagged to identify segments that did not incorporate the tagged nucleotides.

26. The machine readable storage medium of claim 23 that comprises instructions for (a) scanning a surface containing polynucleotides incorporating said detectable nucleotides to obtain signals from said polynucleotides; wherein the polynucleotide is further contacted with one or more detectable marker probes that bind to predefined sequences in a polynucleotide and detecting signals from said bound detectable marker probes.

27. A method for detecting, characterizing or analyzing DNA replication patterns comprising using the machine readable storage medium of claim 23 to receive, process and/or analyze signals from detectably labeled polynucleotides.

28. A method for standardizing, monitoring, and/or verifying reconstruction of one or more genetic modifications that have occurred in or been induced into a genome comprising detecting and analyzing DNA replication patterns of at least one genetically-modified DNA sequence according to claim 1.

29. The method of claim 28, further comprising comparing DNA replication patterns of the at least one genetically-modified DNA sequence with those of an unmodified DNA sequence or control sequence.
